(19) United States
(12) Patent Application Publication
(10) Pub. No.: US 2024/0350564 A1
(43) Pub. Date: Oct. 24, 2024

(54) Composition with NRF Mitochondria Activating Ingredients, Which Has Telomere Extension, Anti-Aging, and Cell Rejuvenating Effects

(71) Applicant: Yu-Lin Yang, Kaohsiung City (TW)

(72) Inventor: Yu-Lin Yang, Kaohsiung City (TW)

(21) Appl. No.: 18/302,922

(22) Filed: Apr. 19, 2023

(51) Int. Cl. A61K 35/745; A61K 35/747; A61K 36/287; A61K 36/41; A61K 36/42; A61K 36/48; A61K 36/537; A61K 36/605; A61K 36/73; A61K 36/736; A61P 39/00

(52) U.S. Cl. CPC A61K 35/745; A61K 35/747; A61K 36/287; A61K 36/41; A61K 36/42; A61K 36/48; A61K 36/537; A61K 36/605; A61K 36/73; A61K 36/736; A61P 39/00; A61K 2035/115

(57) ABSTRACT

A composition with NRF mitochondria activating ingredient has telomere extension, anti-aging and cell-rejuvenating effects, including salvia miltiorrhiza extract, wheat α-amylase inhibitor, and probiotic powder. After taken by users, the composition reaches a health effect by extending the telomere length and to reduce the cell age and get younger.

### BACKGROUND OF THE INVENTION

### Field of the Invention

The present invention relates to a healthy food and, more particularly, to a composition with NRF mitochondria activating ingredients, which has telomere extension, anti-aging, and cell rejuvenating effects.

### Description of the Related Art

Telomeres are repetitive DNA sequences at the end of cell chromosomes. The main effects of the telomeres are to protect the integrity of chromosomes, to ensure the cell replication ability, and to determine the cell division cycle. It is considered that the telomere length is positively related to the health and youthfulness of cells. Therefore, when telomeres are gradually exhausted, the cells will also get aged and die accordingly.

The methods for extending telomeres include adequate sleep, regular exercise, and proper stress relief. However, there is currently no health food or composition that can extend telomeres on the market.

### BRIEF SUMMARY OF THE INVENTION

According to the primary advantages of the present invention, a health composition is provided to improve the health of cells by activating mitochondria and extending telomeres.

For achieving the above-mentioned object, there is a composition with NRF mitochondria activating ingredients, which has telomere extension, anti-aging, and cell rejuvenating effects, comprising:
a salvia miltiorrhiza extract, a wheat α-amylase inhibitor, and probiotic powder which are mixed according to a predetermined weight proportion. The probiotic powder includes Bifidobacterium animalis subsp. lactis and Lactobacillus rhamnosus.

The salvia miltiorrhiza extract contains NRF mitochondrial activating ingredients. Through special active compound extraction technology including herbal chip and HPLC etc., the active ingredients are extracted and concentrated, and experiments have proved that the extract can increase ATP energy molecules in cells (in general, the active ingredients from an ordinary salvia miltiorrhiza extraction methods have limited effects), can activate mitochondria, and can inhibit generation and accumulation of fat. The wheat α-amylase inhibitor can significantly interrupt the conversion of starch into sugars and stabilize blood sugar. The probiotic powder can improve the microbial phase of intestinal tract, increase high-density lipoprotein, and reduce low-density lipoprotein. Aging is caused by multiple causes, including aged mitochondria, a high-sugar diet, and unbalanced gut microbiome. At present, there is no innovative anti-aging combination formula that has triple effects of activating mitochondria, blocking sugar absorption, and reducing fat at the same time. Therefore, an experiment was applied on the 20 subjects who takes the supplement 4 g daily for 2 months. Before and after the experiments, we measure the telomere length from oral mucosal smear, we found that the composition can effectively and statistically significantly extend the telomere length to reduce the cell age, thereby improving health of the human body.

### BRIEF DESCRIPTION OF THE SEVERAL VIEWS OF THE DRAWING(S)

No drawing is provided.

### DETAILED DESCRIPTION OF THE INVENTION

In accordance with the preferred embodiment of the present invention, a composition with NRF mitochondria activating ingredients has telomere extension, anti-aging, and cell rejuvenating effects.

The composition comprises a salvia miltiorrhiza extract with a weight proportion of 1% to 25%, a wheat α-amylase inhibitor with a weight proportion of 5% to 45%, and probiotic powder with a weight proportion of 4% to 30%. The probiotic powder includes Bifidobacterium animalis subsp. lactis with a weight proportion of 50% and Lactobacillus rhamnosus with a weight proportion of 50%.

In the composition, the salvia miltiorrhiza extracted from state-of-art technology is used to activate mitochondria, the wheat α-amylase inhibitor is used to reduce sugar absorption and cut sugar, and the probiotic powder is used to reduce fat. Preferably, the salvia miltiorrhiza extract includes tanshinone, salvianolic acid A, salvianolic acid B, Danshensu, salvianolic acid, silymarin, salvianol, salvianoline, and tanshinazine.

In another preferred embodiment of the present invention, the composition further comprises an alfalfa extract with a weight proportion of 0.5% to 1%. The alfalfa extract is used to activate mitochondria as well.

In another preferred embodiment of the present invention, the composition further comprises a Momordica charantia extract, a fructus mume extract, a strawberry extract, a Rhodiola rosea extract, a Chrysanthemum extract, and a mulberry leaf extract with a weight proportion of 1% to 2%. The Momordica charantia extract, the fructus mume extract, the strawberry extract, the Rhodiola rosea extract, the Chrysanthemum extract, and the mulberry leaf extract are used to reduce sugar and cut sugar.

The following table 1 shows the results of 20 subjects who take the composition at a dosage of 4 grams per day, wherein oral mucosal smears are picked to extract cellular DNA and to amplify by QPCR technology to measure the telomere length before and after two months:

TABLE 1

| | telemore | telemore | telomere | | | telomere |
|---|---|---|---|---|---|---|
| age of | length | length | length | telemore | telemore | age |
| subjects | (before) | (after) | variation (Kb) | age (before) | age (after) | variation |
| 52 | 1.8 | 1.9 | 0.1 | 80 | 79 | −1 |
| 37 | 3.1 | 3.4 | 0.3 | 68 | 65 | −3 |
| 35 | 3.1 | 4.5 | 1.4 | 68 | 54 | −14 |
| 67 | 6.1 | 7.4 | 1.3 | 38 | 25 | −13 |
| 51 | 4.5 | 5.9 | 1.4 | 54 | 40 | −14 |
| 51 | 6.5 | 7.0 | 0.5 | 34 | 30 | −4 |
| 47 | 4.3 | 5.1 | 0.8 | 56 | 48 | −8 |
| 34 | 5.3 | 8.1 | 2.8 | 46 | 18 | −28 |
| 65 | 2.5 | 6.9 | 4.4 | 74 | 30 | −44 |
| 44 | 4.2 | 5.0 | 0.8 | 57 | 49 | −8 |
| 55 | 2.5 | 2.9 | 0.4 | 74 | 70 | −4 |
| 40 | 3.5 | 3.4 | −0.1 | 64 | 65 | 1 |
| 35 | 3.4 | 4.5 | 1.1 | 65 | 54 | −11 |
| 65 | 3.5 | 3.9 | 0.4 | 64 | 60 | −4 |
| 28 | 8.1 | 8.2 | 0.1 | 19 | 18 | −1 |
| 48 | 4.4 | 4.4 | 0.0 | 55 | 55 | 0 |
| 45 | 5.7 | 7.8 | 2.1 | 42 | 22 | −20 |
| 46 | 4.5 | 4.0 | −0.5 | 54 | 59 | 5 |
| 32 | 5.1 | 8.1 | 3.0 | 48 | 18 | −30 |
| 60 | 4.0 | 5.1 | 1.1 | 59 | 48 | 11 |

The test results show that the telomeres from the subjects who take the supplements are extended by an average of 1.074 Kb (kilo base pairs), which proves that the composition of the present invention effectively prolong the telomere length of the subjects, to reduce the age of telomeres (i.e., cellular age), thereby achieving anti-aging and rejuvenating purposes.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the scope of the invention.

1. A composition with NRF mitochondria activating ingredients, which has telomere extension, anti-aging, and cell rejuvenating effects, comprising:
a salvia miltiorrhiza extract with a weight proportion of 1% to 25%, a wheat α-amylase inhibitor with a weight proportion of 5% to 45%, and probiotic powder with a weight proportion of 4% to 30%;
wherein:
the probiotic powder includes Bifidobacterium animalis subsp. lactis with a weight proportion of 50% and Lactobacillus rhamnosus with a weight proportion of 50%.

2. The composition as claimed in claim 1, wherein the composition further comprises an alfalfa extract with a weight proportion of 0.5% to 1%.

3. The composition as claimed in claim 1, wherein the composition further comprises a Momordica charantia extract, a fructus mume extract, a strawberry extract, a Rhodiola rosea extract, a Chrysanthemum extract, and a mulberry leaf extract with a weight proportion of 1% to 2%.
